(19) United States
(12) Patent Application Publication
(10) Pub. No.: US 2023/0348924 A1
(43) Pub. Date: Nov. 2, 2023

(54) USE OF RNA TO TREAT XYLELLA FASTIDIOSA INFECTION IN PLANTS

(71) Applicant: SILVEC BIOLOGICS INC., Gaithersburg, MD (US)

(72) Inventors: Jose Pedro FONSECA, Gaithersburg, MD (US); Anne Elizabeth SIMON, Bowie, MD (US)

(21) Appl. No.: 18/309,979

(22) Filed: May 1, 2023

Related U.S. Application Data
(60) Provisional application No. 63/337,308, filed on May 2, 2022.

(51) Int. Cl. C12N 15/82

(52) U.S. Cl. CPC C12N 15/8218; C12N 15/8281

(57) ABSTRACT

The specification describes an RNA vector. The vector may be derived from a plant virus or an umbravirus-like associated RNA such as Citrus yellow vein-associated virus (CYVaV). The vector contains a heterologous segment comprising a small interfering RNA (siRNA) that targets Xylella fastidiosa (Xf). In some examples, the heterologous segment may target the Xf PilG and/or TolC gene. The heterologous segment may be in the form of a hairpin structure inserted into a CYVaV-like molecule. A method of treating a plant against a disease caused by Xf includes introducing the vector into a plant before or after the plant is infected by Xf. The specification also describes a plant, or a portion of a plant, containing the vector and/or the heterologous segment. The plant may be a grapevine.

### RELATED APPLICATIONS

This application claims the benefit of U.S. Provisional Application 63/337,308 filed on May 2, 2022, which is incorporated by reference herein.

### STATEMENT REGARDING FEDERALLY SPONSORED RESEARCH OR DEVELOPMENT

This invention was made with government support under Grant Number 2035639 awarded by the US National Science Foundation (NSF). The United States government has certain rights in the invention.

### INCORPORATION OF SEQUENCE LISTING

A computer readable form of the Sequence Listing “30059-P71301US01_SequenceListing.xml” (5,162 bytes), was created on Apr. 28, 2023, is filed herewith by electronic submission and is incorporated by reference herein.

### FIELD

This disclosure related to an RNA containing a therapeutic agent and to the treatment of a bacterial infection in a plant.

### BACKGROUND

International Publication Number WO 2020/102210 A1, Plant Vectors, Compositions and Uses Related Thereto, describes a single stranded RNA vector suitable for introducing a therapeutic agent, such as a peptide, a protein or a small RNA, into a host plant. The vector does not encode for any movement protein or coat protein, but is capable of systemic and phloem-limited movement and replication within a host plant.

### INTRODUCTION

The specification describes an RNA vector. The vector may be derived from a plant virus or an umbravirus-like associated RNA. The vector contains a heterologous segment comprising a small interfering RNA (siRNA). The siRNA targets Xylella fastidiosa (Xf). In some examples, the heterologous segment may target the Xf PilG or TolC gene. In some examples, the vector is derived from Citrus yellow vein-associated virus (CYVaV). The heterologous segment may be in the form of a hairpin structure inserted into a CYVaV-like molecule.

This specification further describes a method of treating a plant against a disease caused by Xf by introducing the vector into the plant. The vector may be introduced before or after the plant is infected by Xf.

The specification also describes a plant, or a portion of a plant, containing the vector and/or the heterologous segment.

This specification also describes a method of introducing an RNA vector into grapevine comprising stabbing stems and/or branches of dormant grapevine with a stainless-steel insect pin, followed by injection of a vector agrobacterium suspension.

This specification also describes an interfering RNA targeted against Xf, for example against expression of the PilG or TolC gene of Xf.

### BRIEF DESCRIPTION OF FIGURES

FIG. 1. siRNAs targeting Xf ADK and PilG do not reduce growth of Xf in cell cultures. A. siRNAs purified after RNaselll digestion of dsRNA template PilG and ADK. B. Xf cell cultures grown in vitro one day after addition of 50 ng/μL of siRNAs targeting PilG, ADK and negative controls GFP, H₂O and no template control (NTC).

FIG. 2. Detection of Xf by qPCR from symptomatic (yellow/necrotic) and asymptomatic (green) leaf tissue from Xf-infected N. benthamiana. Histograms represent means of four biological replicates. The expression level was normalized to the EF1 gene from N. benthamiana. Error bars represent se. Asterisk represent statistically significant differences according to Student's t test (P-value<0.05).

FIG. 3. N. benthamiana plants agroinfiltrated with TRV2:PilG from Xf can affect Xf-related disease symptoms. Three weeks-old N. benthamiana plants were agroinfiltrated with TRV2 VIGS vectors carrying Xf genes to be silenced (TRV2:PilG, TRV2:TolC and TRV2:ADK), empty vector (TRV2:00) and water (no VIGS vector control). After 3-weeks, plants were inoculated with Xf. A. Pictures of Xf-infected plants taken at 10-weeks post-inoculation of Xf. TRV2:PilG infected plants display reduced disease symptoms (necrosis and yellowing) compared with the vector alone (TRV2:00) and no VIGS control. B. qPCR with ftsY qPCR primer (Chen et al., 2019) shows reduced expression of Xf ftsY transcript for TRV2:PilG, and TRV2:TolC compared with TRV2:ADK in relation to the empty vector control (TRV2:00). Interestingly, all virus infections reduced Xf levels. Total RNA was isolated from 10-week-old plants for cDNA synthesis. Histograms represent means of four biological replicates. The expression level was normalized to the EF1 gene from N. benthamiana. Error bars represent standard error. Lowercase letters represent statistically significant differences according to Student's t test (P-value<0.05).

FIG. 4. Detection of full length CYVaV from grapevine systemic leaves by RT-PCR at six-weeks post-infiltration. A. Full length CYVaV (˜2.6 kb) RT-PCR amplification from systemic leaves of one of four infiltrated plants by detection of minus (replication intermediate) strands. B. Full length CYVaV (˜2.6 kb) PCR amplification from cDNA synthesized from total RNA extracted from systemic leaves of plant 1 from A. CYVaV control is PCR amplification from CYVaV plasmid.

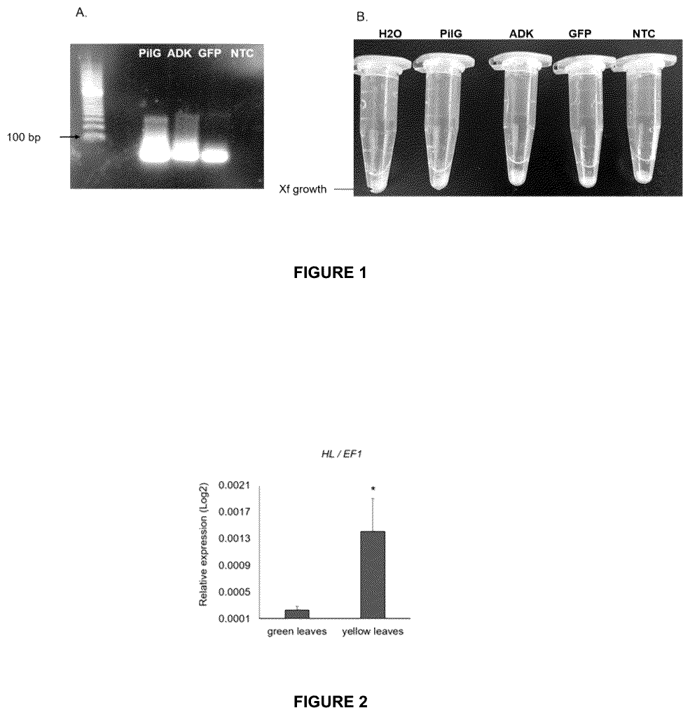

### DETAILED DESCRIPTION

This specification describes various siRNAs that can target Xylella fastidiosa (Xf). The siRNAs can be delivered by a VIGS-like (virus-induced gene silencing) RNA or vector. The vector is demonstrated to silence Xf in the xylem of a plant, for example Nicotiana benthamiana. This specification also describes an inoculation process to introduce a vector into grapevines. The introduced vector was detected in systemic tissue and new leaves (including minus strands) not present during the inoculation, indicating that the vector is moving systemically and replicating in the plant. These results demonstrate that Xf can be targeted in a host plant, for example a tree or vine, using an RNA or viral vector either to immunize or otherwise treat the plant. The treatment does not involve genetic modification of the plant. Combining this work with prior developments indicates that a CYVaV-like vector containing siRNA heterologous elements, for example hairpin or other inserts, will be able to target Xf in grapevine and other host plants.

We generated two different siRNAs for targeting Xf cultures in vitro: one siRNA targeted adenylate kinase (ADK), an enzyme that regulates cellular ATP levels in bacteria (Thach et al., 2014), and the second targeted the pathogenesis-related gene PilG, previously shown to inhibit PD symptoms in grapes when mutated in Xf (Shi and Lin., 2016). We designed primers to amplify the full coding sequence of ADK and PilG from Xf genomic DNA that could be transcribed in both directions by T7 RNA polymerase. The transcribed dsRNA product was then digested with RNase III to generate siRNA products around 20 nt in length. The resulting siRNAs were purified and 50 ng/μL was used to treat overnight Xf cultures. While we did not observe any growth reductions in Xf in these experiments, as shown further below Xf can be targeted successfully in plants.

The colonization of the xylem by Xf is dependent on its ability to move inside the xylem vessels mainly through type IV pili (Mattic., 2002). Xf relies on its twitching motility to move and previous studies indicated that alterations in the PilG gene results in a defective type IV pili and non-twitching phenotypes (Shi and Lin., 2016). In a recent report, no Pierce Disease (PD) symptoms were observed in grapevines inoculated with Xf carrying a mutated PilG gene, although bacterial titers were not significantly altered (from Shi and Lin., 2018). Similarly, mutation of the TolC gene in Xf also resulted in the complete loss of pathogenicity on grapevine (Reddy et al., 2007). TolC is a multidrug resistance efflux pump that traverse both the periplasm and plasma membrane and also a type I-dependent secretion of degradative enzymes and effectors (Reddy et al., 2007). Both PilG and TolC gene products were shown to be required for pathogenicity in grapes infected with Xf (Shi and Lin., 2018; Reddy et al., 2007).

To confirm that model laboratory plant N. benthamiana could be used as a model plant for Xf infection, we inoculated Xf using a novel, adapted version of the pinpricking (“stabbing”) method (Reddy et al., 2007). Symptoms were observed 8 weeks later and Xf levels were quantified in infected tissue by quantitative real-time PCR (qPCR). N. benthamiana leaves inoculated with Xf using the stabbing method displayed leaf yellowing and necrosis. DNA samples from symptomatic (yellow) and asymptomatic (green) leaf samples were collected and significantly more Xf DNA was amplified from symptomatic leaves (yellow) compared to asymptomatic ones (green) (FIG. 2), as previously shown (Francis et al., 2006).

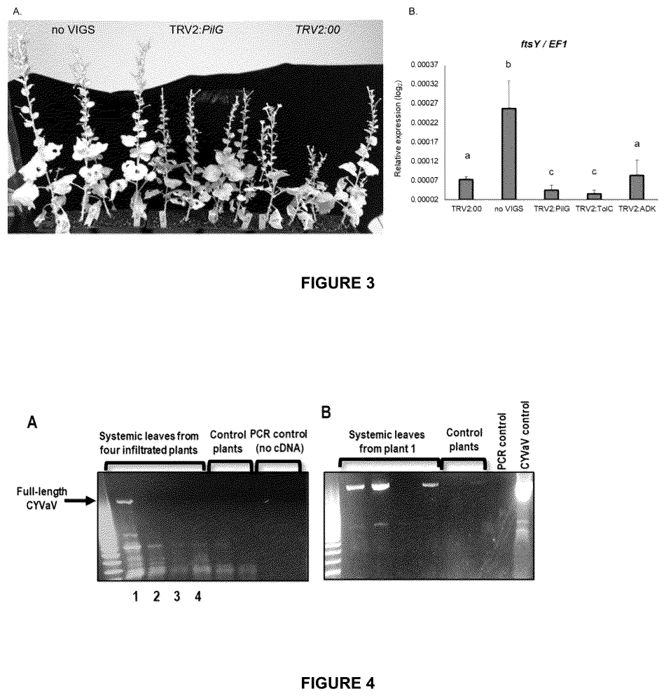

To determine if siRNAs can target Xf in infected N. benthamiana, we cloned three Xf genes (Table 1) into the TRV2 vector and agroinfiltrated into N. benthamiana. Out of the three genes targeted by siRNAs (PilG, TolC and ADK), PilG showed the promising phenotype, reducing Xf disease severity in N. benthamiana (FIG. 3A). This result demonstrates that PilG can be used as a target gene to generate tolerant grape plants against Xf. We also performed qPCR using the ftsY transcript from Xf (Chen et al., 2019) as a proxy for quantification of Xf levels from all samples. We also found significantly less Xf in the TRV2:PilG, TRV2:TolC VIGS-infiltrated plants compared to control plants (TRV2:00) (FIG. 3B). TRV2:ADK VIGS-infiltrated plants displayed similar Xf transcript levels as the empty vector TRV2:00 control (FIG. 3B). Taken together, these data provide proof of concept that Xylella can be targeted by siRNAs in N. benthamiana.

TABLE 1
Candidate genes cloned into TRV2 VIGS vector
for pathogen assays in N. benthamiana.

| Gene | Annotation | Type | Reference |
|---|---|---|---|
| PilG | chemotaxis regulator, motility, and | pathogenesis- | Xi and Li, |
| | signal transduction. | related | 2018 |
| TolC | multidrug resistance efflux system. | pathogenesis- | Reddy et al., |
| | | related | 2006 |
| ADK | adenine nucleotide metabolism and | essential gene | Thach et al., |
| | cellular energy homeostasis. | | 2014 |

Three siRNA of various lengths targeting the Xf PilG gene were found to be effective. The siRNA were cloned into RTV2 vector regions 1, 2 and 3. Regions 2 and 3 are inside of region 1. The sequences of these siRNA are given below. Alternatively, portions or fragments of these sequences may be used. The nucleotide sequences of interfering RNA targeted against regions of the Xf PilG gene are presented below, wherein “t” denotes uracil pursuant to WIPP st.26 standards for sequence listings.

Interfering RNA targeting Region 1 of Xf PilG
gene:
(SEQ ID NO: 1)
GTGCGCATGAGGATATTGGCAATGATTAAAAGTGTTGCTAGCGGCAAGGA
ACTCGCAGGTCTTAGGGTGATGGTCATTGATGACTCAAAAACCATAAGAC
GTACCGCTGAAACGCTTCTTAAGCGTGAAGGGTGTGAGGTGGTTACCGCT
ATTGATGGCTTTGAAGCCTTAGCGAAAATTGCTGATCAGAAGTCGCAGAT
TATTTTTGTCGATATTATGATGCCGCGCTTGGATGGTTATCAAACTTGCG
CGTTGATAAAAAACAATAACTTGTTTAAGTCGACTCCAGTGATCATGCTT
TCTTCTAAAGATGGCTTATTCGATAAGGCGCGCGGTCGTGTGGTTGGTTC
CGAACAATATCTGACCAAACCTTTTACACGCGAGGAGTTGTTAAGTGCCA
TCCGTACATATGTTAATCCTTTAAAATTAGCTGTTTAA
Interfering RNA targeting Region 2 of Xf PilG
gene:
(SEQ ID NO: 2)
GTGCGCATGAGGATATTGGCAATGATTAAAAGTGTTGCTAGCGGCAAGGA
ACTCGCAGGTCTTAGGGTGATGGTCATTGATGACTCAAAAACCATAAGAC
GTACCGCTGAAACGCTTCTTAAGCGTGAAGGGTGTGAGGTGGTTACCGCT
ATTGATGGCTTTGAAGCCTTAGCGAAAATTGCTGATCAGAAGTCGCAGAT
TATTTTTGTCGATATTATGATGCCGCGCTTGGATGGTTATCAAACTTGCG
CGTTGATAAAAAACAATAACTTGTTTAAGTCGACTCCAGTGATCATGCTT
Interfering RNA targeting Region 3 of Xf Pilg Gene
(SEQ ID NO: 3)
GTGCGCATGAGGATATTGGCAATGATTAAAAGTGTTGCTAGCGGCAAGGA
ACTCGCAGGTCTTAGGGTGATGGTCATTGATGACTCAAAAACCATAAGAC

Grapevine is not a natural host for CYVaV, which has only been found in citrus (Kwon et al., 2021; Liu et al., 2021). For CYVaV to work as an efficient VIGS vector in grapevine, it must be able to transit in and out of the phloem sieve tubes and into companion cells and phloem parenchyma cells where the iRNA replicates. Because iRNAs like CYVaV do not express capsid proteins, they are unusually easy to damage ex vivo, which limits inoculation options. In addition, there is limited information on agroinfiltration methods of trees and vines as compared to model plants, and we have found many trees and vines to be incompatible with standard agroinfiltration methods. Fortunately, we were able to agroinfiltrate CYVaV into mature grapevines (Vitis mars) using a modification of the pinpricking method (Yepes et al., 2018). Briefly, stems and branches of dormant grapevine were stabbed with a stainless-steel insect pin, followed by injection on 10 μL of a CYVaV agrobacterium suspension. Six weeks later, RNA was extracted from emerging new leaves and PCR performed to detect CYVaV. As shown in FIG. 4, CYVaV was detected in systemic leaves from inoculated plants. Although CYVaV is more concentrated in roots and new flush, siRNAs released from the silencing of CYVaV by the host plant travel freely throughout a plant. Bacteria must first take up siRNAs for down-regulating gene expression. A 2019 report in BioRxiv demonstrated that host-derived siRNAs can suppress gene expression in phytopathogenic bacteria when applied in vitro and by HIGS (host-induced gene silencing) (https://doi.org/10.1101/863902). Since Xf is known to have the capacity to take up nucleic acids from its environment (Kandel et al., 2016), and because the BioRxiv study also used a gram-negative bacterium, the inventors believe that siRNA released by a CYVaV-like vector will reduce the pathogenicity of Xf.

As described above, Xf targeting siRNAs can be introduced into grapevine using a CYVaV-like vector. A similar approach may be used in olives and other trees infected by Xf.

The heterologous segment, or a portion of it, may alternatively be included in a vector derived from an umbravirus-like associated RNA (ulaRNA). Some ulaRNAs are described in Structural Analysis and Whole Genome Mapping of a New Type of Plant Virus Subviral RNA: Umbravirus-Like Associated RNAs, Liu J. et al., Viruses 2021, 13, 646, which is incorporated herein by reference. As indicated in the title, ulaRNAs may be subviral. However, they are capable of movement and infection in plants. One suitable ulaRNAs is citrus yellow vein associated virus (CYVaV). Although CYVaV is rarely found in nature, it has a broad host range and rarely causes material symptoms of infection. CYVaV may also require a helper virus to move between plants. Since this help virus is not found outside of citrus trees, when CYVaV is used in non-citrus plants it is highly unlikely to spread unintentionally between plants. Alternatively, another ulaRNA, for example a ulaRNA that naturally infects a plant to be treated for Xf infection, may be used.

The use of CYVaV is further described in International Publication Number WO 2020/102210 A1, Plant Vectors, Compositions and Uses Related Thereto, and in International Publication Number WO 2021/097086 A1, Plant Vectors, Compositions and Uses Related Thereto, both of which are incorporated herein by reference. Optionally, a portion of the siRNA described above is converted to a siRNA hairpin, the siRNA portion being one side of the hairpin. The hairpin may be inserted, for example, at position 2250, 2301, 2319, 2330, 2331, 2336 and/or 2375 of CYVaV. The hairpin may have a length on one side of, for example, up to 35 nt or up to 30 nt. The sequence of CYVaV is presented as SEQ ID NO:1 in International Publication Number WO 2021/097086 A1, Plant Vectors, Compositions and Uses Related Thereto.

Optionally, a relative or derivative of CYVaV, or an engineered or synthetic RNA similar to CYVaV (collectively called a CYVaV-like RNA) may be used with a heterologous element comprising an siRNA. A CYVaV-like RNA may have one or more of a) 50% or more or 70% or more RdRp (i.e. SEQ ID NO:8) identify with CYVaV, and b) one or more of SEQ ID NO:2, 3, 4, 5, 6, 10, 11, 12, 13, 14, 15, 16, 17, 18 and 19. Sequence numbers in this paragraph refer to sequence numbers in International Publication Number WO 2021/097086 A1, Plant Vectors, Compositions and Uses Related Thereto.

Optionally, a double stranded RNA (dsRNA) may be made that targets the PilG or TolC gene of Xf. In some examples, a dsRNA similar to the heterologous segment as described above is manufactured outside of the plant and used without the replicating vector. The dsRNA may be introduced onto or into a plant, for example, by foliar spray (e.g. as in spray induced gene silencing, SIGS), by phloem injection or by root uptake. The dsRNA is optionally incorporated into a nanoparticle. The dsRNA may include a portion of SEQ ID NO: 1, SEQ ID NO: 2, or SEQ ID NO: 3 that is at least 19 nt, or at least 21 nt, long on a first side and a complementary sequence on a second side.

1. An RNA comprising,
an RNA comprising all or a portion of a plant virus or an umbravirus-like associated RNA or a relative or derivative therefor, or an similar engineered to synthetic RNA; and,
a heterologous segment comprising a small interfering RNA (siRNA), wherein the siRNA targets Xylella fastidiosa (Xf).

2. The RNA of claim 1 wherein the heterologous segment targets the Xf PilG and/or TolC gene.

3. The RNA of claim 1 wherein the RNA comprises all or a portion of a relative or derivative of CYVaV, or an engineered or synthetic RNA similar to CYVaV.

4. The RNA of claim 1 wherein the heterologous segment is in the form of a hairpin structure.

5. A method of treating a plant against a disease caused by Xf comprising introducing an RNA according to claim 1 into the plant.

6. The method of claim 5 wherein the RNA is introduced before the plant is infected by Xf.

7. The method of claim 5 wherein the RNA is introduced after the plant is infected by Xf.

8. The method of any of claim 5 wherein the plant is a grapevine.

9. A plant, or a portion of a plant, containing the RNA or the heterologous segment of any of claim 1.

10. The plant, or portion of a plant, of claim 9 comprising grapevine.

11. A method of introducing an RNA vector into grapevine comprising stabbing stems and/or branches of dormant grapevine with a stainless-steel insect pin, followed by injection of a vector agrobacterium suspension.

12. An interfering RNA targeted and/or capable of silencing expression of the PilG or TolC gene of Xf.

13. The interfering RNA of claim 12 comprising at least 19 nt, or at least 21 nt, of any of SEQ ID No: 1.

14. The interfering RNA of claim 13 comprising at least 19 nt, or at least 21 nt, of SEQ ID No: 2 or SEQ ID NO: 3.


SEQUENCE LISTING (3 sequences)

SEQ ID NO: 1 - RNA, 438 nt, synthetic construct
  source 1..438: mol_type = other RNA; note = Interfering RNA targeting a region of the Xylella fastidiosa PilG gene
gtgcgcatgaggatattggcaatgattaaaagtgttgctagcggcaaggaactcgcaggtcttagggtgatggtcattgatgactcaaaaaccataagacgtaccgctgaaacgcttcttaagcgtgaagggtgtgaggtggttaccgctattgatggctttgaagccttagcgaaaattgctgatcagaagtcgcagattatttttgtcgatattatgatgccgcgcttggatggttatcaaacttgcgcgttgataaaaaacaataacttgtttaagtcgactccagtgatcatgctttcttctaaagatggcttattcgataaggcgcgcggtcgtgtggttggttccgaacaatatctgaccaaaccttttacacgcgaggagttgttaagtgccatccgtacatatgttaatcctttaaaattagctgtttaa

SEQ ID NO: 2 - RNA, 300 nt, synthetic construct
  source 1..300: mol_type = other RNA; note = Interfering RNA targeting a region of the Xylella fastidiosa PilG gene
gtgcgcatgaggatattggcaatgattaaaagtgttgctagcggcaaggaactcgcaggtcttagggtgatggtcattgatgactcaaaaaccataagacgtaccgctgaaacgcttcttaagcgtgaagggtgtgaggtggttaccgctattgatggctttgaagccttagcgaaaattgctgatcagaagtcgcagattatttttgtcgatattatgatgccgcgcttggatggttatcaaacttgcgcgttgataaaaaacaataacttgtttaagtcgactccagtgatcatgctt

SEQ ID NO: 3 - RNA, 100 nt, synthetic construct
  source 1..100: mol_type = other RNA; note = Interfering RNA targeting a region of the Xylella fastidiosa PilG gene
gtgcgcatgaggatattggcaatgattaaaagtgttgctagcggcaaggaactcgcaggtcttagggtgatggtcattgatgactcaaaaaccataagac